(19) United States
(12) Patent Application Publication
(10) Pub. No.: US 2026/0311879 A1
(43) Pub. Date: Oct. 8, 2026

(54) NEUROLOGICAL DISORDER CLASSIFICATION USING LOG-REGULARIZED LAPLACIAN SPARSE FUNCTIONAL CONNECTIVITY AND ENSEMBLE GRAPH-BASED DEEP LEARNING

(71) Applicant: Texas State University, San Marcos, TX (US)

(72) Inventors: Mylène C. Queiroz de Farias, San Marcos, TX (US); Juliana Mantebea Danso, San Marcos, TX (US)

(21) Appl. No.: 19/638,457

(22) Filed: Apr. 3, 2026

Related U.S. Application Data
(60) Provisional application No. 63/782,983, filed on Apr. 3, 2025.

(51) Int. Cl. G16H 50/20; G06N 20/10; G06N 20/20; G16H 10/60; G16H 30/40

(52) U.S. Cl. CPC G16H 50/20; G06N 20/10; G06N 20/20; G16H 10/60; G16H 30/40

(57) ABSTRACT

A computer-implemented method, system, and computer program product for classifying a neurological disorder. Resting-state functional magnetic resonance imaging (rs-fMRI) data and phenotypic data associated with a subject are received. A sparse functional connectivity (FC) matrix is then estimated from the rs-fMRI data by minimizing an objective function comprising a Laplacian graph trace term and a log-regularized weight term. The discriminative brain connectivity features are then generated from the sparse FC matrix using an Adaptive-Hierarchical Graph Convolutional Network (AH-GCN). Discriminative brain connectivity features refer to a set of high-level, reduced-dimensional latent representations that capture the most topologically significant functional patterns in the subject's brain. Phenotypic data may then be processed through a plurality of machine learning classifiers to generate phenotypic probability scores. Furthermore, the discriminative brain connectivity features and the phenotypic probability scores are synthesized via a stacking meta-classifier to generate a classification for the neurological disorder.

### TECHNICAL FIELD

The present disclosure relates generally to the field of neuroscience and machine learning, and more particularly to diagnosing neurological disorders, such as autism spectrum disorder (ASD), by integrating phenotypic data and sparse functional connectivity (FC) matrices derived from functional magnetic resonance imaging (fMRI) data.

### BACKGROUND

The fields of neuroscience and machine learning have become increasingly intertwined as researchers seek to develop computational models capable of decoding complex brain activity. Machine learning, and specifically graph-based deep learning, offers a powerful framework for analyzing the high-dimensional data generated by neuroimaging. Central to this intersection is the use of functional connectivity (FC) matrices, which represent the statistical dependencies between regional time series in the brain. These matrices serve as a fundamental tool in neuroscience for investigating brain communication and diagnosing various neurological disorders. Traditionally, Pearson Correlation (PC) has been the primary performance metric used to estimate these connections from functional magnetic resonance imaging (fMRI) data. While widely adopted, these conventional methods typically produce dense functional connectivity matrices where every brain region is connected to every other region.

Such dense matrices present significant technical challenges for downstream analysis, particularly when used as inputs for graph-based machine learning models, such as Graph Neural Networks (GNNs). Specifically, dense matrices often contain a high volume of spurious connections that do not reflect actual biological signaling, making the resulting models highly sensitive to noise. Furthermore, the processing of these dense structures results in substantial computational inefficiency and high memory requirements, which can impede real-time clinical diagnostics.

To mitigate these issues, researchers often employ sparsification techniques, such as manual thresholding, to remove weak or irrelevant connections. However, thresholding is frequently an arbitrary and manual process that lacks a rigorous mathematical foundation. Improperly selected thresholds can lead to the loss of critical biological information or fail to sufficiently reduce the noise inherent in the fMRI signal.

Moreover, many existing diagnostic frameworks focus exclusively on neuroimaging data, effectively treating fMRI signals in isolation from other relevant clinical indicators. This unimodal approach fails to leverage the complementary nature of phenotypic data, such as behavioral assessments and demographic information, which can provide essential context for accurate disease classification. Consequently, there is a need for an automated, robust method for estimating sparse functional connectivity that integrates seamlessly with multimodal data to provide more accurate and computationally efficient classification of neurological disorders.

### SUMMARY

In one embodiment of the present disclosure, a computer-implemented method for classifying a neurological disorder comprises receiving resting-state functional magnetic resonance imaging (rs-fMRI) data and phenotypic data associated with a subject. The method further comprises estimating a sparse functional connectivity (FC) matrix from the rs-fMRI data by minimizing an objective function comprising a Laplacian graph trace term and a log-regularized weight term, where the log-regularized weight term comprises a logarithmic barrier to support positive node degrees. The method additionally comprises generating discriminative brain connectivity features from the sparse FC matrix using an Adaptive-Hierarchical Graph Convolutional Network (AH-GCN). Furthermore, the method comprises processing the phenotypic data through a plurality of machine learning classifiers to generate phenotypic probability scores. Additionally, the method comprises synthesizing the discriminative brain connectivity features and the phenotypic probability scores using an ensemble stacking model to generate a classification for the neurological disorder.

Other forms of the embodiment of the computer-implemented method described above are in a system and in a computer program product.

The foregoing has outlined rather generally the features and technical advantages of one or more embodiments of the present disclosure in order that the detailed description of the present disclosure that follows may be better understood. Additional features and advantages of the present disclosure will be described hereinafter which may form the subject of the claims of the present disclosure.

### BRIEF DESCRIPTION OF THE DRAWINGS

A better understanding of the present disclosure can be obtained when the following detailed description is considered in conjunction with the following drawings, in which:

FIG. 1 illustrates an embodiment of the present disclosure of a computing environment for practicing the principles of the present disclosure;

FIG. 2 is a diagram of the software components used by the computer to diagnose neurological disorders in accordance with an embodiment of the present disclosure;

FIG. 3 is a flowchart of a method for diagnosing neurological disorders in accordance with an embodiment of the present disclosure;

FIG. 4 is a detailed block diagram illustrating the general workflow of the multimodal ensemble framework in accordance with an embodiment of the present disclosure; and

FIG. 5 is a detailed workflow diagram illustrating the adaptive brain graph construction process in accordance with an embodiment of the present disclosure.

### DETAILED DESCRIPTION

As stated above, the fields of neuroscience and machine learning have become increasingly intertwined as researchers seek to develop computational models capable of decoding complex brain activity. Machine learning, and specifically graph-based deep learning, offers a powerful framework for analyzing the high-dimensional data generated by neuroimaging. Central to this intersection is the use of functional connectivity (FC) matrices, which represent the statistical dependencies between regional time series in the brain. These matrices serve as a fundamental tool in neuroscience for investigating brain communication and diagnosing various neurological disorders. Traditionally, Pearson Correlation (PC) has been the primary performance metric used to estimate these connections from functional magnetic resonance imaging (fMRI) data. While widely adopted, these conventional methods typically produce dense functional connectivity matrices where every brain region is connected to every other region.

Such dense matrices present significant technical challenges for downstream analysis, particularly when used as inputs for graph-based machine learning models, such as Graph Neural Networks (GNNs). Specifically, dense matrices often contain a high volume of spurious connections that do not reflect actual biological signaling, making the resulting models highly sensitive to noise. Furthermore, the processing of these dense structures results in substantial computational inefficiency and high memory requirements, which can impede real-time clinical diagnostics.

To mitigate these issues, researchers often employ sparsification techniques, such as manual thresholding, to remove weak or irrelevant connections. However, thresholding is frequently an arbitrary and manual process that lacks a rigorous mathematical foundation. Improperly selected thresholds can lead to the loss of critical biological information or fail to sufficiently reduce the noise inherent in the fMRI signal.

Moreover, many existing diagnostic frameworks focus exclusively on neuroimaging data, effectively treating fMRI signals in isolation from other relevant clinical indicators. This unimodal approach fails to leverage the complementary nature of phenotypic data, such as behavioral assessments and demographic information, which can provide essential context for accurate disease classification. Consequently, there is a need for an automated, robust method for estimating sparse functional connectivity that integrates seamlessly with multimodal data to provide more accurate and computationally efficient classification of neurological disorders.

The embodiments of the present disclosure provide a means for improving the accuracy and computational efficiency of neurological disorder classification through a multimodal ensemble framework. At the core of this framework is the automated estimation of sparse functional connectivity (FC) matrices from resting-state functional magnetic resonance imaging (rs-fMRI) data. Rather than relying on arbitrary manual thresholding, the present disclosure utilizes a log-regularized Laplacian Sparse Functional Connectivity (LSFC) method. By introducing a specific regularization term into a Laplacian graph—comprising a logarithmic barrier and a Frobenius norm—the system directly estimates a sparse structure that suppresses spurious connections and noise while preserving the underlying biological signals for diagnostic accuracy.

Furthermore, embodiments of the present disclosure provide an integrated deep learning architecture that leverages both neuroimaging and phenotypic data. An Adaptive-Hierarchical Graph Convolutional Network (AH-GCN) is employed to extract discriminative features from the estimated LSFC matrices. The AH-GCN utilizes multiple adaptive branches to learn edge weights and similarity matrices, coupled with hierarchical clustering to capture multi-scale topological features of the brain. Simultaneously, phenotypic data, such as demographic information and behavioral assessments, is processed through a plurality of machine learning classifiers. This dual-stream approach ensures that the structural and functional nuances of the brain are contextualized by the subject's clinical and personal profile.

To finalize the classification, the outputs from the graph-based deep learning stream and the phenotypic classification stream are synthesized using an ensemble stacking model. By employing a meta-classifier, such as logistic regression, the system optimally weights the contributions of each modality to produce a final diagnostic output, such as an Autism Spectrum Disorder (ASD) classification. This ensemble approach not only achieves superior predictive performance compared to unimodal or dense-matrix methods but also reduces the memory and time overhead required for high-dimensional neuroimaging analysis, thereby facilitating more scalable and reliable clinical decision support. A further discussion regarding these and other features is provided below.

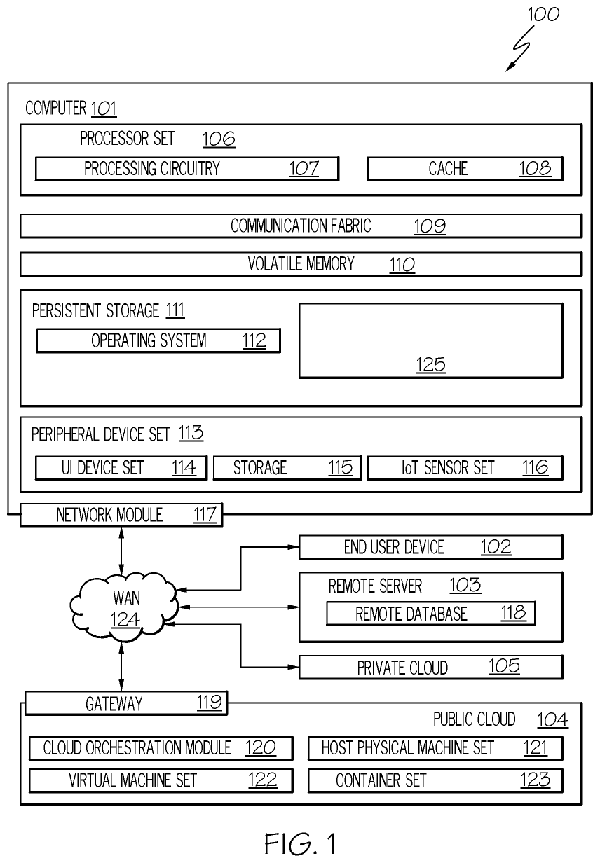

Referring now to the Figures in detail, FIG. 1 illustrates an embodiment of the present disclosure of a computing environment 100 for practicing the principles of the present disclosure.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code (stored in block 125) involved in performing the inventive methods, such as diagnosing neurological disorders. In addition to block 125, computing environment 100 includes, for example, computer 101 (also referred to herein as computing device 101), network 124, such as a wide area network (WAN), end user device (EUD) 102, remote server 103, public cloud 104, and private cloud 105. In this embodiment, computer 101 includes processor set 106 (including processing circuitry 107 and cache 108), communication fabric 109, volatile memory 110, persistent storage 111 (including operating system 112 and block 125, as identified above), peripheral device set 113 (including user interface (UI) device set 114, storage 115, and Internet of Things (IoT) sensor set 116), and network module 117. Remote server 103 includes remote database 118. Public cloud 104 includes gateway 119, cloud orchestration module 120, host physical machine set 121, virtual machine set 122, and container set 123.

Computer 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 118. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

Processor set 106 includes one, or more, computer processors of any type now known or to be developed in the future. Processing circuitry 107 may be distributed over multiple packages, for example, multiple, coordinated integrated circuit chips. Processing circuitry 107 may implement multiple processor threads and/or multiple processor cores. Cache 108 is memory that is located in the processor chip package(s) and is typically used for data or code that should be available for rapid access by the threads or cores running on processor set 106. Cache memories are typically organized into multiple levels depending upon relative proximity to the processing circuitry. Alternatively, some, or all, of the cache for the processor set may be located “off chip.” In some computing environments, processor set 106 may be designed for working with qubits and performing quantum computing.

Computer readable program instructions are typically loaded onto computer 101 to cause a series of operational steps to be performed by processor set 106 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 108 and the other storage media discussed below. The program instructions, and associated data, are accessed by processor set 106 to control and direct performance of the inventive methods. In computing environment 100, at least some of the instructions for performing the inventive methods may be stored in block 125 in persistent storage 111.

Communication fabric 109 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

Volatile memory 110 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 110 is located in a single package and is internal to computer 101, but, alternatively or additionally, the volatile memory may be distributed over multiple packages and/or located externally with respect to computer 101.

Persistent Storage 111 is any form of non-volatile storage for computers that is now known or to be developed in the future. The non-volatility of this storage means that the stored data is maintained regardless of whether power is being supplied to computer 101 and/or directly to persistent storage 111. Persistent storage 111 may be a read only memory (ROM), but typically at least a portion of the persistent storage allows writing of data, deletion of data and re-writing of data. Some familiar forms of persistent storage include magnetic disks and solid state storage devices. Operating system 112 may take several forms, such as various known proprietary operating systems or open source Portable Operating System Interface type operating systems that employ a kernel. The code included in block 125 typically includes at least some of the computer code involved in performing the inventive methods.

Peripheral device set 113 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 114 may include components such as a display screen, speaker, microphone, wearable devices (such as goggles and smart watches), keyboard, mouse, printer, touchpad, game controllers, and haptic devices. Storage 115 is external storage, such as an external hard drive, or insertable storage, such as an SD card. Storage 115 may be persistent and/or volatile. In some embodiments, storage 115 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 116 is made up of sensors that can be used in Internet of Things applications. For example, one sensor may be a thermometer and another sensor may be a motion detector.

Network module 117 is the collection of computer software, hardware, and firmware that allows computer 101 to communicate with other computers through WAN 124. Network module 117 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 117 are performed on the same physical hardware device. In other embodiments (for example, embodiments that utilize software-defined networking (SDN)), the control functions and the forwarding functions of network module 117 are performed on physically separate devices, such that the control functions manage several different network hardware devices. Computer readable program instructions for performing the inventive methods can typically be downloaded to computer 101 from an external computer or external storage device through a network adapter card or network interface included in network module 117.

WAN 124 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

End user device (EUD) 102 is any computer system that is used and controlled by an end user (for example, a customer of an enterprise that operates computer 101), and may take any of the forms discussed above in connection with computer 101. EUD 102 typically receives helpful and useful data from the operations of computer 101. For example, in a hypothetical case where computer 101 is designed to provide a recommendation to an end user, this recommendation would typically be communicated from network module 117 of computer 101 through WAN 124 to EUD 102. In this way, EUD 102 can display, or otherwise present, the recommendation to an end user. In some embodiments, EUD 102 may be a client device, such as thin client, heavy client, mainframe computer, desktop computer and so on.

Remote server 103 is any computer system that serves at least some data and/or functionality to computer 101. Remote server 103 may be controlled and used by the same entity that operates computer 101. Remote server 103 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 118 of remote server 103.

Public cloud 104 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 104 is performed by the computer hardware and/or software of cloud orchestration module 120. The computing resources provided by public cloud 104 are typically implemented by virtual computing environments that run on various computers making up the computers of host physical machine set 121, which is the universe of physical computers in and/or available to public cloud 104. The virtual computing environments (VCEs) typically take the form of virtual machines from virtual machine set 122 and/or containers from container set 123. It is understood that these VCEs may be stored as images and may be transferred among and between the various physical machine hosts, either as images or after instantiation of the VCE. Cloud orchestration module 120 manages the transfer and storage of images, deploys new instantiations of VCEs and manages active instantiations of VCE deployments. Gateway 119 is the collection of computer software, hardware, and firmware that allows public cloud 104 to communicate through WAN 124.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

Private cloud 105 is similar to public cloud 104, except that the computing resources are only available for use by a single enterprise. While private cloud 105 is depicted as being in communication with WAN 124 in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 104 and private cloud 105 are both part of a larger hybrid cloud.

Block 125 further includes the software components which are used to diagnose neurological disorders. In one embodiment, such components may be implemented in hardware. The functions discussed above performed by such components are not generic computer functions. As a result, computer 101 is a particular machine that is the result of implementing specific, non-generic computer functions.

In one embodiment, the functionality of such software components of computer 101, including the functionality for diagnosing neurological disorders, may be embodied in an application specific integrated circuit.

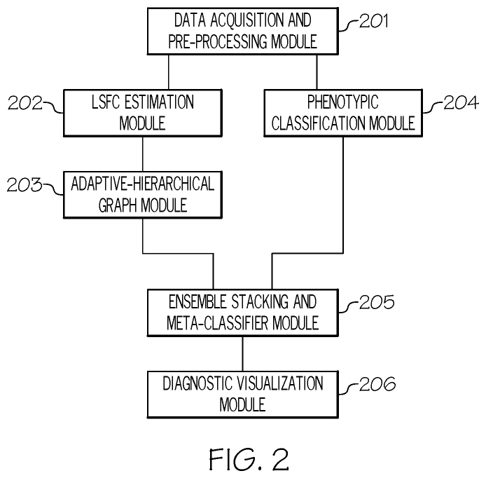

A further discussion regarding the functionality of the components used by computer 101 to diagnose neurological disorders is provided below in connection with FIG. 2.

FIG. 2 is a diagram of the software components used by computer 101 to diagnose neurological disorders in accordance with an embodiment of the present disclosure.

Referring now to FIG. 2, in conjunction with FIG. 1, computer 101 includes data acquisition and pre-processing module 201. In one embodiment, data acquisition and pre-processing module 201 is configured to receive and prepare the disparate data modalities required for neurological classification. Specifically, data acquisition and pre-processing module 201 receives resting-state functional magnetic resonance imaging (rs-fMRI) data, which includes blood-oxygen-level-dependent (BOLD) signals representing brain activity over time, and phenotypic data, which includes non-imaging subject information, such as age, gender, and clinical behavioral assessment scores (e.g., ADOS, ADI-R).

To ensure the integrity of the downstream analysis, data acquisition and pre-processing module 201 performs a series of standardized pre-processing operations on the rs-fMRI data. These operations may include, but are not limited to, slice-timing correction, motion correction, nuisance regression (e.g., global signal regression), and spatial smoothing. Additionally, in one embodiment, data acquisition and pre-processing module 201 is configured to perform brain parcellation, where the brain is divided into a plurality of distinct regions of interest (ROIs) based on a predefined anatomical or functional atlas. The resulting regional time-series signals are then formatted for functional connectivity estimation. Simultaneously, data acquisition and pre-processing module 201 cleans and normalizes the phenotypic data, ensuring that categorical and numerical features are appropriately scaled for processing by the machine learning classifiers. By centralizing these preparatory steps, data acquisition and pre-processing module 201 ensures that the subsequent modules receive high-quality, synchronized inputs, thereby reducing the risk of noise-driven errors in the final diagnostic classification.

Computer 101 further includes LSFC (Log-Regularized Laplacian Sparse Functional Connectivity) estimation module 202 configured to automate the direct estimation of a sparse functional connectivity matrix from the pre-processed rs-fMRI data. Unlike conventional methods that generate dense matrices requiring arbitrary thresholding, LSFC estimation module 202 utilizes a log-regularized Laplacian graph framework to identify a sparse structure that preserves high-signal-to-noise ratio (SNR) connections while suppressing spurious links. In one embodiment, LSFC estimation module 202 receives the regional time-series signals from data acquisition and pre-processing module 201 and constructs a connectivity matrix by solving an optimization problem that minimizes an objective function defined by:

$${min}_{L} trace ( {X}^{T} LX ) + ℛ ( W )$$

where X represents the rs-fMRI signals, L is the Laplacian matrix, W is the weight associated with L, and [symbol] (W) is a specialized weight regularization term.

Specifically, the weight regularization term [symbol] (W) implemented by LSFC estimation module 202 includes a logarithmic barrier designed to support positive node degrees, effectively ensuring that each brain region remains connected to at least one other region in the network. Furthermore, the regularization term includes a Frobenius norm of the weight matrix. The Frobenius norm operates to penalize excessively large connectivity values and promote uniform sparsity across the sparse FC matrix. By integrating these specific mathematical constraints, LSFC estimation module 202 generates a sparse FC matrix that is significantly more robust to motion artifacts and global signal resonance (GSR) effects than standard Pearson Correlation-based matrices. The output of LSFC estimation module 202 is a high-fidelity, sparse representation of the subject's unique neural architecture, which serves as the input for the subsequent graph-based deep learning analysis.

Computer 101 additionally includes adaptive-hierarchical graph module 203 configured to extract discriminative brain connectivity features from the sparse FC matrix generated by LSFC estimation module 202. Discriminative brain connectivity features, as used herein, refers to a set of high-level, reduced-dimensional latent representations that capture the most topologically significant functional patterns in the subject's brain.

In one embodiment, adaptive-hierarchical graph module 203 implements an Adaptive-Hierarchical Graph Convolutional Network (AH-GCN) architecture that dynamically adjusts the graph structure during the learning process. In one embodiment, adaptive-hierarchical graph module 203 includes three parallel processing branches: (i) a similarity-adaptive branch that generates an adaptive similarity matrix (S) using a softmax-normalized Gaussian kernel; (ii) a structural branch that computes a symmetric normalized adjacency matrix (Anorm); and (iii) a weight-adaptive branch that learns adaptive edge weights (W′) through a fully connected layer (FCL) and a masking operation.

By synthesizing the outputs of these three branches, adaptive-hierarchical graph module 203 constructs an adaptive brain graph that better represents the underlying functional topology than a static graph. In one embodiment, adaptive-hierarchical graph module 203 constructs an adaptive brain graph by synthesizing an adaptive similarity matrix, a normalized adjacency matrix, and a set of learned edge weights. An adaptive similarity matrix, as used herein, refers to a mathematical representation (e.g., matrix S) that characterizes the proximity or statistical alignment between node features. In one embodiment, the adaptive similarity matrix is generated using a softmax-normalized Gaussian kernel to embed node features from an initial K-nearest neighbors graph (KNNG), thereby rendering the resulting graph adaptive to the specific connectivity strengths between regions of interest (ROIs) in the subject's brain. A normalized adjacency matrix, as used herein, refers to a standardized structural representation (e.g., matrix Aₙₒᵣₘ) of the brain graph that accounts for node degrees. In one embodiment, the normalized adjacency matrix is a symmetric matrix computed from the adjacency and degree matrices of the KNNG, ensuring that the scale of the graph features remains consistent across successive layers of the neural network. A set of learned edge weights, as used herein, refers to a vector or matrix of optimized connectivity values (e.g., weights W) that are learned during the training process of the AH-GCN. By synthesizing these three components and passing the result through a nonlinear activation function, adaptive-hierarchical graph module 203 produces a set of final adaptive brain graph features. This synthesized representation allows the framework to dynamically adjust to the subject's unique neural architecture, providing a significantly more robust input for the hierarchical clustering and classification stages of the framework.

Furthermore, adaptive-hierarchical graph module 203 is configured to perform hierarchical clustering (or hierarchical pooling) on the adaptive brain graph. This hierarchical operation allows the system to reduce the dimensionality of the high-resolution brain graph while simultaneously extracting multi-scale topological features that characterize the global and local organization of the subject's brain. The resulting feature vectors, which encode the most salient connectivity patterns for disorder classification, are then provided to the ensemble stacking meta-classifier for final synthesis.

Furthermore, computer 101 includes phenotypic classification module 204 configured to process non-imaging data associated with the subject to generate phenotypic probability scores. Phenotypic probability scores, as used herein, refer to the quantitative outputs generated by a plurality of machine learning classifiers, where each score represents a statistical likelihood or confidence level that the subject possesses a specific neurological condition (e.g., a score of 0.85 indicating an 85% probability of ASD).

While neuroimaging data provides a functional snapshot of the brain, phenotypic data, such as age, gender, and clinical assessment scores, provides critical context that significantly improves the specificity of the diagnostic model. In one embodiment, phenotypic classification module 204 utilizes a plurality of machine learning (ML) classifiers to process these features in parallel. For example, phenotypic classification module 204 may implement a Support Vector Machine (SVM) to find an optimal hyperplane for classification, a Random Forest (RF) classifier to handle non-linear decision boundaries through an ensemble of decision trees, and a gradient boosting framework (e.g., CatBoost) to optimize prediction accuracy through sequential error correction.

By utilizing a diversity of ML classifiers, phenotypic classification module 204 captures different statistical nuances within the phenotypic data. Each classifier within phenotypic classification module 204 outputs a probability score representing the likelihood that the subject belongs to a specific diagnostic category (e.g., ASD vs. typically developing). These scores are then aggregated and passed to ensemble stacking and meta-classifier module 205, ensuring that the final diagnostic decision is informed not only by the brain's functional architecture but also by the subject's established clinical and demographic profile.

Computer 101 additionally includes ensemble stacking and meta-classifier module 205 configured to synthesize the multimodal outputs into a final classification for the neurological disorder. In one embodiment, ensemble stacking and meta-classifier module 205 functions as a “meta-learner” that receives two primary sets of inputs: (i) the discriminative brain connectivity feature vectors from adaptive-hierarchical graph module 203; and (ii) the phenotypic probability scores from phenotypic classification module 204.

Specifically, ensemble stacking and meta-classifier module 205 implements a stacking architecture where a meta-classifier, such as a logistic regression (LR) model, is trained to optimally weight the predictions from the various sub-modules. This synthesis compensates for the potential weaknesses of any single data modality or classifier. For instance, if the neuroimaging features suggest a borderline case, the meta-classifier may rely more heavily on the phenotypic probability scores to resolve the diagnosis. The final output of ensemble stacking and meta-classifier module 205 is a definitive classification (e.g., a binary ASD/non-ASD result or a multi-class severity rating), which may be transmitted to an end-user device 102 or stored in remote database 118 for clinical review. This ensemble stacking architecture ensures that the structural nuances captured by the AH-GCN and the clinical markers captured by the ML classifiers are mathematically balanced to maximize the Area Under the Receiver Operating Characteristic (AUROC) curve for the final classification.

Additionally, computer 101 includes diagnostic visualization module 206 configured to translate the high-dimensional mathematical outputs of the classification process into human-interpretable clinical insights. While the ensemble stacking and meta-classifier module 205 provides a definitive diagnostic result, diagnostic visualization module 206 provides the “explainability” required for clinical adoption. In one embodiment, diagnostic visualization module 206 is configured to perform an importance analysis (e.g., using saliency maps or integrated gradients) on the AH-GCN to identify which specific brain regions or connectivity edges contributed most significantly to the final classification.

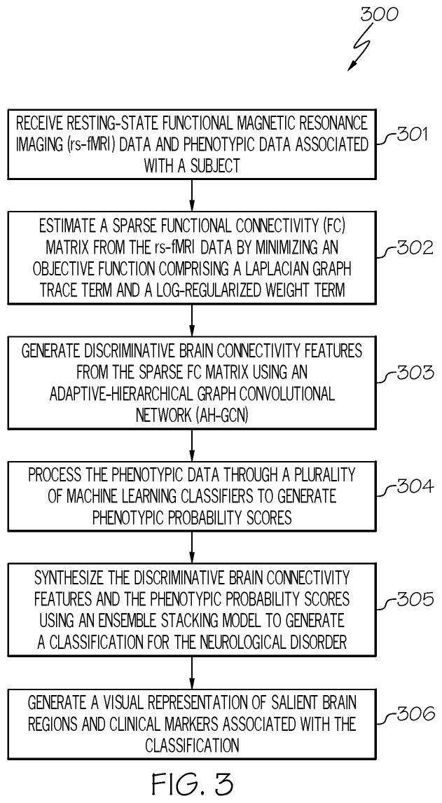

For example, diagnostic visualization module 206 may generate 3D brain renderings that highlight “top salient regions” associated with the neurological disorder, such as the prefrontal cortex or the superior temporal sulcus in cases of ASD. These renderings may be color-coded to indicate the strength or direction of the functional connectivity anomalies compared to a normative population. Furthermore, diagnostic visualization module 206 may generate comparative charts, such as modularity and Small-World Property (SWP) distributions, to illustrate how the subject's sparse FC structure differs from traditional dense-matrix models. By providing these visual and statistical markers, diagnostic visualization module 206 enables a clinician to validate the automated diagnosis against established neuroanatomical markers, thereby serving as a robust decision-support tool. A further discussion regarding the operational steps and logic executed by these modules is provided below in connection with FIG. 3.

FIG. 3 is a flowchart of a method 300 for diagnosing neurological disorders in accordance with an embodiment of the present disclosure.

Referring now to FIG. 3, in conjunction with FIGS. 1-2, in step 301, data acquisition and pre-processing module 201 receives resting-state functional magnetic resonance imaging (rs-fMRI) data and phenotypic data associated with a subject.

Resting-state functional magnetic resonance imaging (rs-fMRI) data, as used herein, refers to a non-invasive neuroimaging modality that measures spontaneous fluctuations in blood-oxygen-level-dependent (BOLD) signals while a subject is at rest and not performing a specific cognitive task. In one embodiment, the rs-fMRI data is received as a four-dimensional (4D) NIfTI file format containing a temporal sequence of three-dimensional (3D) brain volumes. In one embodiment, data acquisition and pre-processing module 201 receives resting-state functional magnetic resonance imaging (rs-fMRI) data associated with a subject by interfacing with a medical imaging database (e.g., a Picture Archiving and Communication System (PACS) or a research repository like ABIDE) via network module 117.

Phenotypic data, as used herein, refers to non-imaging clinical and demographic characteristics that provide context to the neuroimaging signals. This data includes, but is not limited to: demographic markers (e.g., age, gender, ethnicity); behavioral assessment scores (e.g., Autism Diagnostic Observation Schedule (ADOS) scores, Social Responsiveness Scale (SRS) scores); and other clinical indicators. In one embodiment, data acquisition and pre-processing module 201 receives phenotypic data associated with a subject by querying an Electronic Health Record (EHR) system or receiving a structured data file (e.g., a CSV or JSON file) containing the subject's clinical profile.

A subject, as used herein, refers to a human or non-human individual being evaluated for a potential neurological disorder. While embodiments discussed herein focus on pediatric subjects for the classification of Autism Spectrum Disorder (ASD), the term “subject” is intended to encompass individuals across all age groups and clinical conditions involving altered brain connectivity, such as Alzheimer's disease or schizophrenia.

In one embodiment, data acquisition and pre-processing module 201 implements a pre-processing sub-routine to standardize the disparate data formats. For the rs-fMRI data, this may involve using a pre-processing pipeline (e.g., Configurable Pipeline for the Analysis of Connectomes (C-PAC)) to perform motion correction and spatial normalization. For the phenotypic data, this may involve encoding categorical variables (e.g., one-hot encoding for gender) and normalizing numerical variables (e.g., Z-score scaling for age) to ensure compatibility with the downstream machine learning classifiers in phenotypic classification module 204. By integrating these two data streams at the inception of the method, data acquisition and pre-processing module 201 establishes a multimodal foundation that enhances the robustness of the subsequent sparse functional connectivity estimation.

In step 302, LSFC estimation module 202 estimates a sparse functional connectivity (FC) matrix from the rs-fMRI data by minimizing an objective function comprising a Laplacian graph trace term and a log-regularized weight term.

A sparse FC matrix, as used herein, refers to a mathematical representation of the brain's functional organization where a majority of the entries in the matrix are zero, reflecting only the most statistically significant and biologically relevant connections between regions of interest (ROIs). Unlike dense matrices produced by standard Pearson Correlation, the sparse FC matrix estimated in step 302 filters out noise and spurious correlations, resulting in a cleaner graph structure that is computationally efficient for downstream neural network processing.

A Laplacian graph trace term, as used herein, refers to a mathematical constraint that enforces “smoothness” on the estimated graph. In one embodiment, this term is expressed as trace (X^{T}LX), where X is the matrix of rs-fMRI signals and L is the Laplacian matrix. Minimizing this term ensures that brain regions with similar temporal BOLD signal profiles are assigned higher connectivity weights, thereby capturing the intrinsic functional co-activation patterns of the subject's brain.

A log-regularized weight term, as used herein, refers to a specialized penalty function applied to the connectivity weights (W) to control the topology of the resulting graph. This term comprises two primary components: (i) a logarithmic barrier (e.g., −Σ log(dᵢ), where d represents node degrees) that prevents nodes from becoming isolated by supporting positive node degrees; and (ii) a Frobenius norm

$$( e . g . , {‖ W ‖}_{F}^{2} )$$

that prevents any single connection from becoming disproportionately large.

In one embodiment, LSFC estimation module 202 estimates the sparse functional connectivity (FC) matrix from the rs-fMRI data by minimizing an objective function comprising a Laplacian graph trace term and a log-regularized weight term by executing an iterative optimization algorithm (such as a primal-dual splitting method or a coordinate descent algorithm) to solve the objective function. LSFC estimation module 202 receives the pre-processed BOLD time-series from step 301 and iteratively updates the weight matrix W until the objective function converges. By automating this process, the method eliminates the need for manual, arbitrary thresholding. The resulting sparse FC matrix provides a robust, high-fidelity input that is less sensitive to motion and global signal artifacts, thereby improving the diagnostic reliability of the overall framework.

In step 303, adaptive-hierarchical graph module 203 generates the discriminative brain connectivity features from the sparse FC matrix using an Adaptive-Hierarchical Graph Convolutional Network (AH-GCN).

Discriminative brain connectivity features, as used herein, refer to a set of high-level, reduced-dimensional latent representations that capture the most topologically significant functional patterns in the subject's brain. These features are “discriminative” in that they emphasize the specific connectivity anomalies (e.g., hyper-connectivity or hypo-connectivity in specific neural circuits) that distinguish a disordered brain state, such as ASD, from a typically developing brain state.

An Adaptive-Hierarchical Graph Convolutional Network (AH-GCN), as used herein, refers to a deep learning architecture designed to perform convolutions on graph-structured data where the underlying graph is not static, but is instead dynamically refined during the training process. The “adaptive” component refers to the network's ability to learn and adjust the strength of connections between nodes based on the data itself, rather than relying on a fixed input graph. The “hierarchical” component refers to the use of graph pooling or clustering operations that aggregate information across multiple scales of the brain's organization.

In one embodiment, adaptive-hierarchical graph module 203 generates these features by executing a multi-branch graph construction process. This process includes: (i) calculating a similarity-adaptive matrix (S) using a Gaussian kernel to measure the proximity of ROI features; (ii) generating a weight-adaptive matrix (W′) via a fully connected layer and a masking operation configured to zero-out or suppress edges below a specific significance threshold to focus on the most relevant edges; and (iii) synthesizing these with a normalized adjacency matrix (Aₙₒᵣₘ) derived from the sparse FC matrix.

In one embodiment, the AH-GCN performs successive layers of graph convolutions followed by hierarchical clustering. During hierarchical clustering, adaptive-hierarchical graph module 203 identifies clusters of ROIs that exhibit similar functional behavior and collapses them into “super-nodes,” effectively reducing the dimensionality of the graph while preserving its global topological properties. This allows the system to extract features that are robust to local variations and noise. By automating the extraction of these multi-scale features, step 303 provides a highly condensed and informative representation of the brain's functional state, which is then used as the primary neuroimaging input for the final ensemble classification.

In step 304, phonotypic classification module 204 processes the phenotypic data through a plurality of machine learning classifiers to generate phenotypic probability scores.

A machine learning classifier, as used herein, refers to a computational algorithm trained to identify patterns within a dataset and assign input data to one or more discrete categories or continuous values. In the context of the present disclosure, the plurality of machine learning classifiers comprises a diverse set of models—such as Support Vector Machines (SVM), Random Forests (RF), and gradient boosting frameworks (e.g., CatBoost)—each characterized by a different mathematical approach to decision-making. By utilizing a “plurality” of such classifiers, the system captures a broader range of statistical relationships within the phenotypic data than could be achieved by a single model.

Phenotypic probability scores, as used herein, refer to the quantitative outputs generated by the plurality of machine learning classifiers, where each score represents a statistical likelihood or confidence level that the subject possesses a specific neurological condition (e.g., a score of 0.85 indicating an 85% probability of ASD). These scores serve as a standardized numerical representation of the clinical and demographic context, allowing non-imaging data to be mathematically synthesized with the graph-based features extracted in step 303.

In one embodiment, phenotypic classification module 204 processes the phenotypic data through a plurality of machine learning classifiers to generate phenotypic probability scores by executing the plurality of classifiers in a parallel processing architecture. Phonotypic classification module 204 receives the cleaned and normalized phenotypic features (e.g., age, gender scores, and behavioral indices) from step 301. Each classifier—having been previously trained on a labeled dataset, such as the ABIDE repository—independently processes the features to generate its respective probability score. For instance, the SVM may calculate a score based on the subject's position relative to a high-dimensional hyperplane, while the RF classifier calculates a score based on the majority vote of an ensemble of decision trees.

In one embodiment, the performance of each classifier in step 304 is optimized using a hyperparameter tuning process (such as grid search or Bayesian optimization) to ensure maximum sensitivity and specificity for the target neurological disorder. By generating multiple, independent probability scores, the method creates a robust phenotypic profile that mitigates the risk of overfitting inherent in individual machine learning models. These probability scores are then transmitted to ensemble stacking and meta-classifier module 205 for final synthesis.

In step 305, ensemble stacking and meta-classifier module 205 synthesizes the discriminative brain connectivity features and the phenotypic probability scores using an ensemble stacking model to generate a classification for the neurological disorder.

An ensemble stacking model, as used herein, refers to a hierarchical machine learning architecture where the predictions or features generated by a first layer of models (e.g., the AH-GCN of adaptive-hierarchical graph module 203 and the ML classifiers of phenotypic classification module 204) are used as inputs for a second-level “meta-model.” Unlike simple averaging or voting, an ensemble stacking model learns the optimal way to combine these diverse inputs, effectively weighting the imaging-based features against the phenotypic-based scores to minimize overall prediction error.

In one embodiment, the ensemble stacking model utilizes a logistic regression (LR) meta-classifier to weight relative contributions of the AH-GCN and the plurality of machine learning classifiers. A logistic regression (LR) meta-classifier, as used herein, refers to a secondary statistical model that receives a concatenated feature vector—comprising both the latent neuroimaging representations and the phenotypic probability scores—and applies a logistic function to estimate the probability of a categorical outcome (e.g., ASD vs. typically developing). By utilizing an LR meta-classifier, the system is configured to learn a specific set of coefficients (weights) that dictate the “relative contribution” of each input stream. For example, if the neuroimaging features exhibit higher predictive power for a specific subject group, the LR meta-classifier automatically assigns a higher weight to the AH-GCN output, thereby ensuring the final diagnosis is not disproportionately biased by less reliable phenotypic markers, or vice versa.

In one embodiment, ensemble stacking and meta-classifier module 205 implements the ensemble stacking model by utilizing a logistic regression (LR) meta-classifier. The meta-classifier is configured to receive the multi-dimensional feature vector from the AH-GCN and the vector of probability scores from the phenotypic stream. The meta-classifier then applies a learned weighting function to these inputs to calculate a final decision value. This stacking approach is particularly advantageous for neurological disorders, such as ASD, where the biological signals in the brain may be subtle and require the corroboration of clinical phenotypic markers to reach a high-confidence diagnosis.

In one embodiment, ensemble stacking and meta-classifier module 205 synthesizes the discriminative brain connectivity features and the phenotypic probability scores using an ensemble stacking model to generate a classification for the neurological disorder by performing a synthesis operation that aligns the temporal and structural scales of the input data. Specifically, ensemble stacking and meta-classifier module 205 concatenates the brain connectivity features and phenotypic scores into a single unified feature set. In one embodiment, this synthesis operation includes a feature scaling sub-routine (e.g., Min-Max scaling or Standardized scaling) to ensure that the disparate numerical ranges of the connectivity features and the probability scores are normalized for the meta-classifier. The unified feature set is then fed into the meta-classifier. To ensure the robustness of the classification, the meta-classifier may be trained using a cross-validation technique (e.g., leave-one-out or k-fold cross-validation) on a standardized dataset to prevent the final model from becoming biased toward any specific data modality.

The output of step 305 is a definitive classification for the neurological disorder. This classification may be a binary label (e.g., “Positive for ASD” or “Negative for ASD”) or a continuous score indicating the severity of the condition. By automating the synthesis of multimodal data through a stacking meta-classifier, the method achieves a higher Area Under the Receiver Operating Characteristic (AUROC) curve and superior sensitivity compared to single-modality diagnostic tools.

In step 306, diagnostic visualization module 206 generates a visual representation of salient brain regions and clinical markers associated with the classification.

A visual representation, as used herein, refers to a human-interpretable graphical or schematic output that maps the high-dimensional mathematical results of the classification process back onto an anatomical or clinical context. This representation serves to “de-mystify” the deep learning process by highlighting the specific biological and phenotypic indicators that led to the final diagnostic classification.

In one embodiment, diagnostic visualization module 206 generates the visual representation by calculating saliency maps or feature importance weights. For the imaging stream, diagnostic visualization module 206 identifies the nodes and edges in the adaptive brain graph that had the highest impact on the AH-GCN's decision. For the phenotypic stream, diagnostic visualization module 206 identifies which clinical markers (e.g., a specific behavioral score) were most influential in the meta-classifier's final output.

In one embodiment, diagnostic visualization module 206 generates a visual representation of salient brain regions and clinical markers associated with the classification by utilizing a 3D rendering engine or a graphical plotting interface to project the saliency data onto a standardized brain atlas (e.g., the Montreal Neurological Institute (MNI) space). In one embodiment, this projection involves a coordinate transformation that maps the specific regions of interest (ROIs) from the subject's native space to the voxel-based coordinates of the standardized atlas. For instance, diagnostic visualization module 206 may generate a “heat map” of the subject's brain where the regions of interest (ROIs) associated with the disorder are highlighted in contrasting colors. Additionally, diagnostic visualization module 206 may generate a “contribution report” in the form of a bar chart or spider plot, showing the relative weight of the imaging features versus the phenotypic features in the final diagnosis.

The output of step 306 provides a clinician with actionable insights that go beyond a simple “positive” or “negative” label. By visualizing the “top salient regions” (e.g., the frontal and temporal lobes) and identifying the most predictive clinical markers, the method allows for a personalized understanding of the subject's specific neuro-phenotype. This transparency not only aids in clinical validation but also assists in the development of targeted therapeutic interventions based on the identified brain-behavior relationships.

While FIG. 3 illustrates the high-level operational method, a more detailed architectural view of the multimodal integration and the specific data flow paths is provided below.

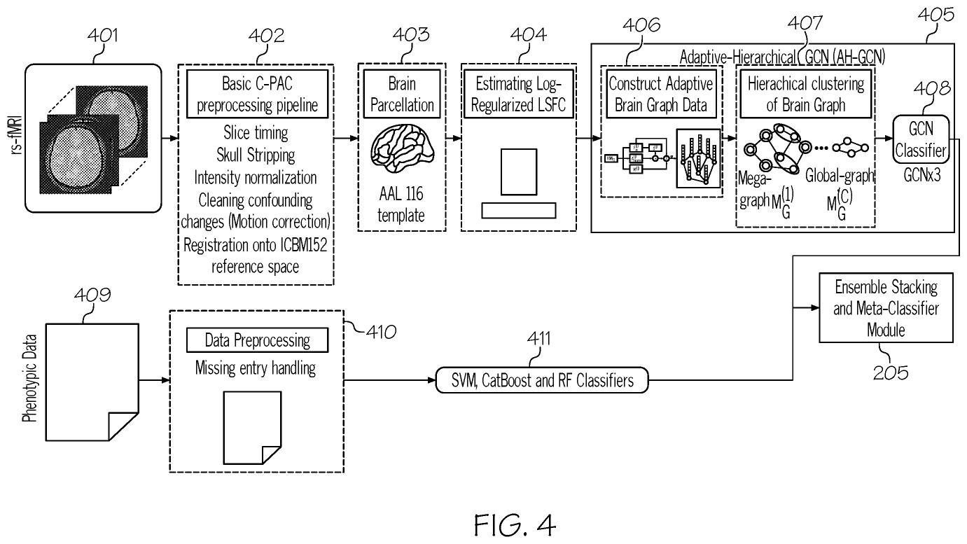

FIG. 4 is a detailed block diagram illustrating the general workflow of the multimodal ensemble framework in accordance with an embodiment of the present disclosure.

Referring now to FIG. 4, in conjunction with FIGS. 1-3, the multimodal ensemble framework 400 illustrates the bifurcated processing of neuroimaging and phenotypic data streams. The neuroimaging stream begins with the acquisition of rs-fMRI data 401. This data is processed through a standard preprocessing pipeline 402 (e.g., C-PAC), which performs motion correction and signal cleaning as previously described. Following preprocessing, a brain parcellation module 403 divides the 4D fMRI volume into a plurality of regions based on a functional atlas to generate regional time-series data.

These regional time-series are then provided to the LSFC estimation block 404, where the log-regularized Laplacian sparse functional connectivity matrix is constructed. This matrix serves as the primary input to the AH-GCN module 405. As shown in FIG. 4, AH-GCN module 405 extracts the discriminative brain connectivity features through a multi-stage adaptive learning process. Specifically, AH-GCN module 405 is configured to first construct adaptive brain graph data (see element 406) by refining the graph topology based on the specific sparse connections estimated in block 404. Once the adaptive graph is constructed, AH-GCN module 405 performs hierarchical clustering of the brain graph (see element 407) to aggregate information across multiple scales and reduce dimensionality while preserving global topological properties. Finally, a GCN classifier 408 within AH-GCN module 405 processes these multi-scale features to output a latent representation of the subject's brain connectivity. This integrated structure allows AH-GCN module 405 to dynamically adjust to the subject's unique neural architecture before passing the extracted features to ensemble stacking and meta-classifier module 205.

Simultaneously, the phenotypic data stream 409 undergoes a data preprocessing step 410, where clinical and demographic variables are normalized and encoded. These prepared features are then fed into a plurality of machine learning (ML) classifiers 411, which in this embodiment include a Support Vector Machine (SVM), a Random Forest (RF), and a CatBoost classifier. Each of these classifiers generates an independent probability score for the target neurological disorder.

The final stage of multimodal ensemble framework 400 is the ensemble stacking and meta-classifier module 205. Ensemble stacking and meta-classifier module 205 receives the latent feature representations from AH-GCN module 405 and the probability scores from ML classifiers 411. A Logistic Regression (LR) meta-classifier of ensemble stacking and meta-classifier module 205 performs a weighted synthesis of these multimodal inputs to produce the final ensemble classification. By visualizing the system in this integrated manner, it is evident how the structural fMRI features and clinical phenotypic markers are mathematically harmonized to achieve superior diagnostic accuracy.

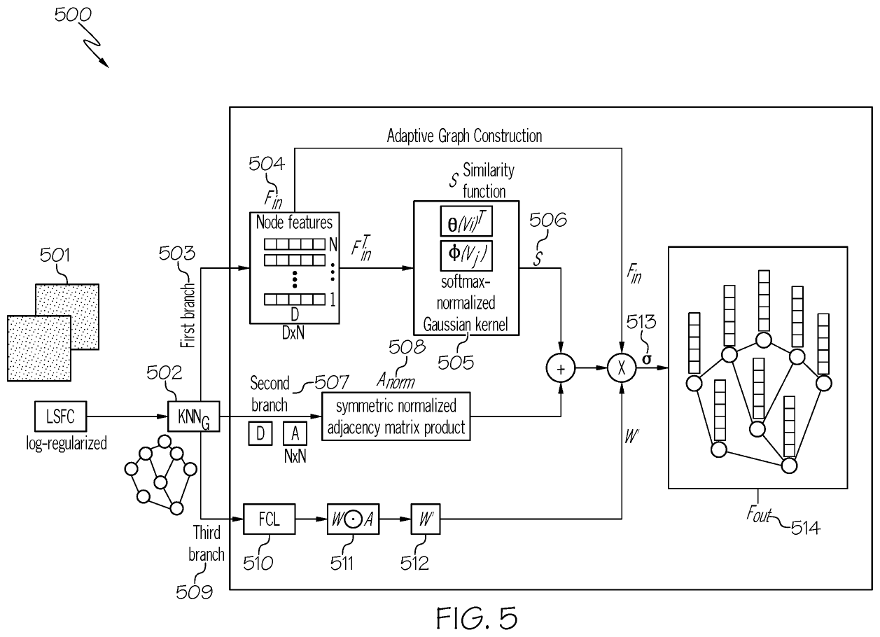

To further detail the internal operations of the adaptive graph construction referenced in step 303 and block 406 of FIG. 4, a detailed view of the triple-branch architecture is provided below in connection with FIG. 5.

FIG. 5 is a detailed workflow diagram illustrating the adaptive brain graph construction process 500 in accordance with an embodiment of the present disclosure.

Referring now to FIG. 5, the process utilizes the log-regularized LSFC matrix 501 to initialize a K-Nearest Neighbors Graph (KNNG) 502. Process 500 then extracts features through three distinct, parallel processing branches to render the graph adaptive to the specific connectivity strengths of the subject.

In a first branch 503, original node features (Fᵢₙ) 504 from KNNG 502 are processed using a softmax-normalized Gaussian kernel 505 to compute a pairwise similarity matrix S 506. This branch utilizes 1×1 convolutional layers to perform distinct transformations, denoted as θ(⋅) and φ(⋅), which render the graph adaptive to the underlying neural signal proximity between nodes (νᵢ, νⱼ).

In a second branch 507, the adjacency and degree matrices of KNNG 502 are used to compute a symmetric normalized adjacency matrix Aₙₒᵣₘ 508. Simultaneously, in a third branch 509, a Fully Connected Layer (FCL) 510 learns new node weights (W). A masking operation 511 denoted as (W⊙Â) is executed between the original adjacency matrix and these learned weights to yield adaptive edge weights W′ 512.

The outputs from these three branches—the similarity matrix S 506, the normalized adjacency matrix Aₙₒᵣₘ 508, and the adaptive weights W′ 512—are synthesized and passed through a nonlinear activation function 513 (denoted as σ). This synthesis produces the final adaptive brain graph features Fₒᵤₜ 514, which are then provided to the subsequent GCN layers for multi-scale feature extraction.

Furthermore, the principles of the present disclosure improve the technology or technical field involving neuroscience and machine learning.

As discussed above, the fields of neuroscience and machine learning have become increasingly intertwined as researchers seek to develop computational models capable of decoding complex brain activity. Machine learning, and specifically graph-based deep learning, offers a powerful framework for analyzing the high-dimensional data generated by neuroimaging. Central to this intersection is the use of functional connectivity (FC) matrices, which represent the statistical dependencies between regional time series in the brain. These matrices serve as a fundamental tool in neuroscience for investigating brain communication and diagnosing various neurological disorders. Traditionally, Pearson Correlation (PC) has been the primary performance metric used to estimate these connections from functional magnetic resonance imaging (fMRI) data. While widely adopted, these conventional methods typically produce dense functional connectivity matrices where every brain region is connected to every other region. Such dense matrices present significant technical challenges for downstream analysis, particularly when used as inputs for graph-based machine learning models, such as Graph Neural Networks (GNNs). Specifically, dense matrices often contain a high volume of spurious connections that do not reflect actual biological signaling, making the resulting models highly sensitive to noise. Furthermore, the processing of these dense structures results in substantial computational inefficiency and high memory requirements, which can impede real-time clinical diagnostics. To mitigate these issues, researchers often employ sparsification techniques, such as manual thresholding, to remove weak or irrelevant connections. However, thresholding is frequently an arbitrary and manual process that lacks a rigorous mathematical foundation. Improperly selected thresholds can lead to the loss of critical biological information or fail to sufficiently reduce the noise inherent in the fMRI signal. Moreover, many existing diagnostic frameworks focus exclusively on neuroimaging data, effectively treating fMRI signals in isolation from other relevant clinical indicators. This unimodal approach fails to leverage the complementary nature of phenotypic data, such as behavioral assessments and demographic information, which can provide essential context for accurate disease classification. Consequently, there is a need for an automated, robust method for estimating sparse functional connectivity that integrates seamlessly with multimodal data to provide more accurate and computationally efficient classification of neurological disorders.

Embodiments of the present disclosure improve such technology by receiving resting-state functional magnetic resonance imaging (rs-fMRI) data and phenotypic data associated with a subject. Resting-state functional magnetic resonance imaging (rs-fMRI) data, as used herein, refers to a non-invasive neuroimaging modality that measures spontaneous fluctuations in blood-oxygen-level-dependent (BOLD) signals while a subject is at rest and not performing a specific cognitive task. Phenotypic data, as used herein, refers to non-imaging clinical and demographic characteristics that provide context to the neuroimaging signals. This data includes, but is not limited to: demographic markers (e.g., age, gender, ethnicity); behavioral assessment scores (e.g., Autism Diagnostic Observation Schedule (ADOS) scores, Social Responsiveness Scale (SRS) scores); and other clinical indicators. A subject, as used herein, refers to a human or non-human individual being evaluated for a potential neurological disorder. A sparse functional connectivity (FC) matrix is then estimated from the rs-fMRI data by minimizing an objective function comprising a Laplacian graph trace term and a log-regularized weight term. A sparse FC matrix, as used herein, refers to a mathematical representation of the brain's functional organization where a majority of the entries in the matrix are zero, reflecting only the most statistically significant and biologically relevant connections between regions of interest (ROIs). Unlike dense matrices produced by standard Pearson Correlation, the estimated sparse FC matrix filters out noise and spurious correlations, resulting in a cleaner graph structure that is computationally efficient for downstream neural network processing. The log-regularized weight term provides a mathematically principled foundation for sparsity that eliminates the need for arbitrary manual thresholding. A Laplacian graph trace term, as used herein, refers to a mathematical constraint that enforces “smoothness” on the estimated graph. A log-regularized weight term, as used herein, refers to a specialized penalty function applied to the connectivity weights (W) to control the topology of the resulting graph. The discriminative brain connectivity features are then generated from the sparse FC matrix using an Adaptive-Hierarchical Graph Convolutional Network (AH-GCN). Discriminative brain connectivity features, as used herein, refers to a set of high-level, reduced-dimensional latent representations that capture the most topologically significant functional patterns in the subject's brain. These features are “discriminative” in that they emphasize the specific connectivity anomalies (e.g., hyper-connectivity or hypo-connectivity in specific neural circuits) that distinguish a disordered brain state, such as ASD, from a typically developing brain state. An Adaptive-Hierarchical Graph Convolutional Network (AH-GCN), as used herein, refers to a deep learning architecture designed to perform convolutions on graph-structured data where the underlying graph is not static, but is instead dynamically refined during the training process. The “adaptive” component refers to the network's ability to learn and adjust the strength of connections between nodes based on the data itself, rather than relying on a fixed input graph. This adaptive capability allows the framework to account for significant inter-subject variability in neural architectures that fixed-graph models cannot capture. The “hierarchical” component refers to the use of graph pooling or clustering operations that aggregate information across multiple scales of the brain's organization. Phenotypic data may then be processed through a plurality of machine learning classifiers to generate phenotypic probability scores. A machine learning classifier, as used herein, refers to a computational algorithm trained to identify patterns within a dataset and assign input data to one or more discrete categories or continuous values. Phenotypic probability scores, as used herein, refer to the quantitative outputs generated by the plurality of machine learning classifiers, where each score represents a statistical likelihood or confidence level that the subject possesses a specific neurological condition (e.g., a score of 0.85 indicating an 85% probability of ASD). Furthermore, the discriminative brain connectivity features and the phenotypic probability scores are synthesized via a stacking meta-classifier to generate a classification for the neurological disorder. In this manner, embodiments of the present disclosure provide a means for improving the accuracy and computational efficiency of neurological disorder classification through a multimodal ensemble framework. Furthermore, in this manner, there is an improvement in the technical field involving neuroscience and machine learning.

The technical solution provided by the present disclosure cannot be performed in the human mind or by a human using a pen and paper. That is, the technical solution provided by the present disclosure could not be accomplished in the human mind or by a human using a pen and paper in any reasonable amount of time and with any reasonable expectation of accuracy without the use of a computer. Specifically, the high-dimensional nature of fMRI time-series data and the complex iterative optimization required for log-regularized sparse estimation and adaptive graph convolution necessitate the specialized processing power of a computational system.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A computer-implemented method for classifying a neurological disorder, the method comprising:
receiving resting-state functional magnetic resonance imaging (rs-fMRI) data and phenotypic data associated with a subject;
estimating a sparse functional connectivity (FC) matrix from said rs-fMRI data by minimizing an objective function comprising a Laplacian graph trace term and a log-regularized weight term, wherein said log-regularized weight term comprises a logarithmic barrier to support positive node degrees;
generating discriminative brain connectivity features from said sparse FC matrix using an Adaptive-Hierarchical Graph Convolutional Network (AH-GCN);
processing said phenotypic data through a plurality of machine learning classifiers to generate phenotypic probability scores; and
synthesizing said discriminative brain connectivity features and said phenotypic probability scores using an ensemble stacking model to generate a classification for said neurological disorder.

2. The method as recited in claim 1, wherein said log-regularized weight term further comprises a Frobenius norm of a weight matrix to penalize large connectivity values and promote uniform sparsity across said sparse FC matrix.

3. The method as recited in claim 1, wherein said generating of said discriminative brain connectivity features using said AH-GCN further comprises:
constructing an adaptive brain graph by synthesizing an adaptive similarity matrix, a normalized adjacency matrix, and a set of learned edge weights.

4. The method as recited in claim 3, wherein said AH-GCN further performs hierarchical clustering on said adaptive brain graph to reduce dimensionality and extract multi-scale topological features of a brain of said subject.

5. The method as recited in claim 1, wherein said plurality of machine learning classifiers comprises at least two of: a Support Vector Machine (SVM), a Random Forest (RF) classifier, and a gradient boosting framework.

6. The method as recited in claim 1, wherein said ensemble stacking model utilizes a logistic regression meta-classifier to weight relative contributions of said AH-GCN and said plurality of machine learning classifiers.

7. The method as recited in claim 1, wherein said neurological disorder is an autism spectrum disorder (ASD), wherein said phenotypic data comprises at least one of: age, gender, and a clinical behavioral assessment score.

8. A computer program product for classifying a neurological disorder, the computer program product comprising one or more computer readable storage mediums having program code embodied therewith, the program code comprising programming instructions for:
receiving resting-state functional magnetic resonance imaging (rs-fMRI) data and phenotypic data associated with a subject;
estimating a sparse functional connectivity (FC) matrix from said rs-fMRI data by minimizing an objective function comprising a Laplacian graph trace term and a log-regularized weight term, wherein said log-regularized weight term comprises a logarithmic barrier to support positive node degrees;
generating discriminative brain connectivity features from said sparse FC matrix using an Adaptive-Hierarchical Graph Convolutional Network (AH-GCN);
processing said phenotypic data through a plurality of machine learning classifiers to generate phenotypic probability scores; and
synthesizing said discriminative brain connectivity features and said phenotypic probability scores using an ensemble stacking model to generate a classification for said neurological disorder.

9. The computer program product as recited in claim 8, wherein said log-regularized weight term further comprises a Frobenius norm of a weight matrix to penalize large connectivity values and promote uniform sparsity across said sparse FC matrix.

10. The computer program product as recited in claim 8, wherein said generating of said discriminative brain connectivity features using said AH-GCN further comprises:
constructing an adaptive brain graph by synthesizing an adaptive similarity matrix, a normalized adjacency matrix, and a set of learned edge weights.

11. The computer program product as recited in claim 10, wherein said AH-GCN further performs hierarchical clustering on said adaptive brain graph to reduce dimensionality and extract multi-scale topological features of a brain of said subject.

12. The computer program product as recited in claim 8, wherein said plurality of machine learning classifiers comprises at least two of: a Support Vector Machine (SVM), a Random Forest (RF) classifier, and a gradient boosting framework.

13. The computer program product as recited in claim 8, wherein said ensemble stacking model utilizes a logistic regression meta-classifier to weight relative contributions of said AH-GCN and said plurality of machine learning classifiers.

14. The computer program product as recited in claim 8, wherein said neurological disorder is an autism spectrum disorder (ASD), wherein said phenotypic data comprises at least one of: age, gender, and a clinical behavioral assessment score.

15. A system, comprising:
a memory for storing a computer program for classifying a neurological disorder; and
a processor connected to the memory, wherein the processor is configured to execute program instructions of the computer program comprising:
receiving resting-state functional magnetic resonance imaging (rs-fMRI) data and phenotypic data associated with a subject;
estimating a sparse functional connectivity (FC) matrix from said rs-fMRI data by minimizing an objective function comprising a Laplacian graph trace term and a log-regularized weight term, wherein said log-regularized weight term comprises a logarithmic barrier to support positive node degrees;
generating discriminative brain connectivity features from said sparse FC matrix using an Adaptive-Hierarchical Graph Convolutional Network (AH-GCN);
processing said phenotypic data through a plurality of machine learning classifiers to generate phenotypic probability scores; and
synthesizing said discriminative brain connectivity features and said phenotypic probability scores using an ensemble stacking model to generate a classification for said neurological disorder.

16. The system as recited in claim 15, wherein said log-regularized weight term further comprises a Frobenius norm of a weight matrix to penalize large connectivity values and promote uniform sparsity across said sparse FC matrix.

17. The system as recited in claim 15, wherein said generating of said discriminative brain connectivity features using said AH-GCN further comprises:
constructing an adaptive brain graph by synthesizing an adaptive similarity matrix, a normalized adjacency matrix, and a set of learned edge weights.

18. The system as recited in claim 17, wherein said AH-GCN further performs hierarchical clustering on said adaptive brain graph to reduce dimensionality and extract multi-scale topological features of a brain of said subject.

19. The system as recited in claim 15, wherein said plurality of machine learning classifiers comprises at least two of: a Support Vector Machine (SVM), a Random Forest (RF) classifier, and a gradient boosting framework.

20. The system as recited in claim 15, wherein said ensemble stacking model utilizes a logistic regression meta-classifier to weight relative contributions of said AH-GCN and said plurality of machine learning classifiers.
